(19) United States
(12) Patent Application Publication
(10) Pub. No.: US 2007/0237796 A1
(43) Pub. Date: Oct. 11, 2007

(54) Stable High Pl Hydrogel Compositions

(76) Inventors: Guyla Vigh, College Station, TX (US); Sanjiv Kumar S. Lalwani, College Station, TX (US)

Correspondence Address: COOLEY GODWARD KRONISH LLP;ATTN: Patent Group, Suite 500, 1200 - 19th Street, NW, WASHINGTON, DC, 20036-2402, US

(21) Appl. No.: 10/561,154

(22) PCT Filed: Jun. 17, 2004
(86) PCT No.: PCT/US04/19631

§ 371 Date: Apr. 2, 2007

Related U.S. Application Data
(60) Provisional application No. 60/478,835, filed on Jun. 17, 2003.

(51) Int. Cl. A61K 9/00

(52) U.S. Cl. 424/400

(57) ABSTRACT

The invention provides a hydrolytically stable isoelectric hydrogel material having a pi value in the range of 11.5<pI<14, that is comprised of an isoelectric compound having a pI value in the range of 11.5<pI<14. The pI of the isoelectric compound and thus, the pI of the hydrolytically stable isoelectric hydrogel material, is conferred by the pI value of the isoelectric compound. The hydrolytically stable isoelectric hydrogel material may further comprise a hydrolytically stable hydrophilic polymer and a cross-linker. The invention also provides a hydrolytically stable isoelectric membrane that comprises the hydrolytically stable isoelectric hydrogel material, and methods for preparing a hydrolytically stable isoelectric membrane.

### CROSS REFERENCES TO RELATED APPLICATIONS

This application is related to U.S. Patent Application No. 60/478,835, filed Jun. 17, 2003, which is herein incorporated by reference in its entirety.

### FIELD OF THE INVENTION

This invention relates to high-pI materials and hydrolytically stable, high-pI hydrogel and/or membrane compositions made from these materials. Additionally, this invention relates to the use of hydrolytically stable, high-pI hydrogel and/or membrane compositions in analytical and preparative-scale isoelectric focusing separation and/or isoelectric trapping separation of ampholytic compounds.

### BACKGROUND OF THE INVENTION

Electrophoretic techniques and isoelectric focusing (IEF) techniques in particular, remain key technologies for the separation of ampholytic components. IEF is a widely used technique that may be performed on either an analytical or a preparative scale. For example, IEF may be used in clinical diagnosis, biotechnology, pharmaceutical and food industries. Furthermore, IEF may be used alone or may be used in combination with other analytical or preparative techniques.

In IEF, ampholytic components are separated with the help of an electric field in a pH gradient wherein the pH increases from a lower pH value at the anode to a higher pH value at the cathode. (For a monograph on IEF, see, e.g., P. G. Righetti, Isoelectric focusing: theory, methodology and applications, Elsevier Biomedical, Amsterdam, 1983, which is herein incorporated by reference). Since the net charge of an ampholytic component is zero in its isoelectric state, the electrophoretic migration velocity of an ampholytic component is zero whenever the pH of its environment becomes equal to its isoelectric point (pI) value. Thus, ampholytic components with different pI values stop migrating at different points in a pH gradient.

Relatively stable continuous pH gradients can be created by several means. For example, mixtures of carrier ampholytes (compounds that have adequate buffering ability and conductivity in the vicinity of their pI value) may be used. Alternatively, appropriate amounts of suitable weak acids and weak bases or weak acids and strong bases or strong acids and weak bases may be bound, in a spatially controlled manner, into an ion-permeable matrix, such as a cross-linked polyacrylamide gel. The bound acids and bases provide a preformed and stabilized the pH gradient which may then be used for immobilized pH gradient IEF (IPGIEF). (For a monograph on IPGIEF, see, e.g., P. G. Righetti, Immobilized pH gradients: theory and methodology, Elsevier, Amsterdam, 1990, which is herein incorporated by reference).

Ampholytic sample components can also be separated from each other by isoelectric trapping (IET) utilizing isoelectric membrane-based multicompartmental electrolyzers (e.g., Faupel et al., U.S. Pat. No. 5,082,548, which is incorporated herein by reference) wherein at the end of an IET separation process, ampholytic sample components are obtained in their isoelectric state.

Despite the versatility of IEF and IET technologies, the present methods are not particularly suitable for separation or processing of compounds having very high isoelectric points, because the separation media presently employed are not particularly stable at extreme pH values. Thus, there is a need in the art for hydrolytically stable, high-pI hydrogel and/or membrane compositions for the separation of compounds having very high pI values. As will become apparent from the following detailed description, the present invention provides hydrolytically stable, high-pI hydrogel and/or membrane compositions to meet this and other needs.

### SUMMARY OF THE INVENTION

The invention provides a hydrolytically stable isoelectric hydrogel material having a pI value in the range of 11.5<pI<14, that is comprised of an isoelectric compound having a pI value in the range of 11.5<pI<14. The pI of the isoelectric compound and thus, the pI of the hydrolytically stable isoelectric hydrogel material, is conferred by the pI value of the isoelectric compound. The hydrolytically stable isoelectric hydrogel material may further comprise a hydrolytically stable hydrophilic polymer and a cross-linker. The invention also provides a hydrolytically stable isoelectric membrane that comprises the hydrolytically stable isoelectric hydrogel material, and methods for preparing a hydrolytically stable isoelectric membrane.

Thus, in a first aspect, the present invention provides a hydrolytically stable isoelectric hydrogel material having a pI value in the range of 11.5<pI<14 (e.g., between 11.5, 12, 12.5, 13, 13.5, and 14), wherein the hydrolytically stable isoelectric hydrogel material comprises an isoelectric compound having a pI value in the range of 11.5<pI<14, wherein the pI is established by a substantially permanently cationic group and two hydroxyl groups with pKₐ values in the range of 11.5<pKₐ<14. The hydrolytically stable isoelectric hydrogel material also comprises an additional reactive group, and the pI value of the isoelectric hydrogel is conferred by the pI value of the isoelectric compound. In another aspect, the hydrolytically stable isoelectric hydrogel material may further comprise a hydrolytically stable hydrophilic polymer comprised of two or more reactive groups that are substantially free of ionic functional groups, together with a cross-linker substantially free of ionic functional groups that comprises two groups able to react with both the isoelectric compound and the hydrolytically stable polymer without altering the pI value of the isoelectric compound by more than about 1 pH unit. In one aspect, the pI value of the isoelectric hydrogel is changed by changing identity of the isoelectric compound.

In another aspect, the isoelectric compound comprising the isoelectric hydrogel material is present at a concentration that is equal to or higher than necessary to create an aqueous solution of the isoelectric compound having pH equal to the pI value of the isoelectric compound.

In another aspect, the isoelectric compound is a native or derivatized carbohydrate or polyhydroxy compound, the permanently cationic functional group is a quaternary ammonium group, the additional reactive group of the isoelectric compound is a hydroxyl group, the hydrolytically stable hydrophilic polymer is an oligosaccharide or a synthetic polymer containing multiple hydroxyl groups, and the cross-linker has two or more reactive groups selected from the group consisting of an aldehyde, epoxy, halo, alkylsulfonyl, and arylsulfonyl groups.

In another aspect, the native or derivatized carbohydrate or polyhydroxy compound comprising the isoelectric compound is selected from the group consisting of native or derivatized monosaccharides, native or derivatized disaccharides, native or derivatized trisaccharides, native or derivatized oligosaccharides, native or derivatized polysaccharides, native or derivatized cyclodextrins, native or derivatized maltodextrins, native or derivatized amyloses, native or derivatized dextrans, native or derivatized starches, native or derivatized celluloses, and native or derivatized guar gums, the quaternary ammonium group comprising the cationic functional group comprises one or more substituents selected from the group consisting of methyl, ethyl, propyl, butyl, higher alkyl, hydroxymethyl, hydroxyethyl, hydroxypropyl, hydroxybutyl, higher hydroxyalkyl, saturated ring systems, and unsaturated ring systems (e.g. aryl, heteroaryl, cycloalkyl, heterocycloalkyl), the hydrolytically stable hydrophilic polymer is selected from the group consisting of hydroxymethyl cellulose, hydroxyethyl cellulose, and poly(vinyl alcohol), and the cross-linker is selected from the group consisting of glutaraldehyde, glycerol-1,3-diglycidyl ether, bis-(2-bromoethylene)-ethyleneglycol, and bis(2-tosylethylene)-ethyleneglycol.

In another aspect the isoelectric compound is a glycidyl alkylammonium derivative such as a glycidyl trimethylammonium derivative of beta-cyclodextrin, the hydrolytically stable hydrophilic polymer is poly(vinyl alcohol), and (c) the cross-linking agent is glycerol-1,3-diglycidyl ether.

The invention also provides a hydrolytically stable isoelectric membrane having a pI value in the range of 11.5<pI<14 comprising a hydrolytically stable isoelectric hydrogel supported on a hydrolytically stable, porous, inert or reactive substrate.

In one aspect the hydrolytically stable isoelectric hydrogel membrane the porous substrate comprising the membrane is selected from the group consisting of poly(vinyl alcohol), partially or fully hydrolyzed poly(epihalohydrin), partially or fully hydrolyzed poly(epihalohydrin-co-ethylene oxide), poly(vinyl sulfone), and poly(ether-ether ketone).

The invention also provides, hydrolytically stable hydrophilic isoelectric hydrogel material having a pI value in the range of 11.5<pI<14 comprising a precursor comprising a hydroxyl group with a pKa value in the range of 11.5<pKa<14 and an additional reactive group, a derivatizing agent comprising a permanently cationic functional group and an additional reactive group, wherein the derivatizing agent is structurally different from the precursor, and wherein the pI value of the isoelectric hydrogel is conferred by the concentration of the derivatizing agent and the precursor and the pKa value of the hydroxyl group of the precursor. In a related aspect, the hydrolytically stable hydrophilic isoelectric hydrogel material may further comprise a hydrolytically stable hydrophilic polymer having two or more reactive groups that is substantially free of ionic functional groups, wherein the hydrophilic polymer agent is structurally different from the precursor and the derivatizing agent, and a cross-linker substantially free of ionic functional groups and having two groups able to react with both the precursor and the hydrolytically stable hydrophillic polymer, wherein the cross-linker is structurally different from both the precursor, the derivatizing agent and the polymer.

In another related aspect, the invention provides a hydrolytically stable hydrophilic isoelectric hydrogel material wherein the precursor is a native or derivatized carbohydrate or polyhydroxy compound, the derivatizing agent comprises a quaternary ammonium group, the reactive group of the derivatizing agent is selected from the group consisting of hydroxyl, aldehyde, epoxy, halo, alkylsulfonyl, and arylsulfonyl groups, the hydrolytically stable hydrophilic polymer is an oligosaccharide or a synthetic polymer containing multiple hydroxyl groups, and the cross-linker has two or more reactive groups selected from the group consisting of aldehyde, epoxy, halo, alkylsulfonyl, and arylsulfonyl groups.

In another related aspect the invention provides a hydrolytically stable hydrophilic isoelectric hydrogel material wherein the precursor is selected from the group consisting of native or derivatized monosaccharides, native or derivatized disaccharides, native or derivatized trisaccharides, native or derivatized oligosaccharides, native or derivatized polysaccharides, native or derivatized cyclodextrins, native or derivatized maltodextrins, native or derivatized amyloses, native or derivatized dextrins, native or derivatized starches, native or derivatized celluloses, and native or derivatized guar gums, the quaternary ammonium group of the derivatizing agent contains one or more substituents selected from the group consisting of methyl, ethyl, propyl, butyl, higher alkyl, hydroxymethyl, hydroxyethyl, hydroxypropyl, hydroxybutyl, higher hydroxyalkyl groups, saturated ring systems, and unsaturated ring systems, the cross-linker is selected from the group consisting of glutaraldehyde, glycerol-1,3-diglycidyl ether, bis-(2-bromoethylene)-ethyleneglycol, and bis(2-tosylethylene)-ethyleneglycol and the hydrolytically stable polymer is selected from the group consisting of hydroxyethyl cellulose, hydroxymethyl cellulose, and poly(vinyl alcohol).

In a related aspect, the precursor is beta-cyclodextrin, the derivatizing agent is glycidyl-trimethylammonium chloride, the cross-linker is glycerol-1,3-diglycidyl ether, and the hydrolytically stable hydrophilic polymer is poly(vinyl alcohol). In a specific embodiment the precursor is poly(vinylalcohol), and the cross-linker is glycerol-1,3-diglycidyl ether and the derivatizing agent is glycidyl-trimethylammonium chloride.

The invention also provides a hydrolytically stable isoelectric membrane having a pI in the range of 11.5<pI<14 comprised of a hydrolytically stable isoelectric hydrogel having a pI in the 11.5<pI<14, supported on a hydrolytically stable, porous, inert or reactive substrate. In one embodiment, the material of the porous substrate is selected from the group consisting of poly(vinyl alcohol), partially or fully hydrolyzed poly(epihalohydrin), partially or fully hydrolyzed poly(epihalohydrin-co-ethylene oxide), poly(vinyl sulfone), and poly(ether-ether ketone).

The invention further provides a method of forming a hydrolytically stable isoelectric membrane having a pI value in the range of 11.5<pI<14, comprising the steps of (a) selecting a carbohydrate-based or polyhydroxy compound-based isoelectric material having a pI value in the 11.5<pI<14 range, (b) reacting the isoelectric material at a concentration sufficiently high to set the pH of its aqueous solution equal to its pI value, with a cross-linker substantially free of ionic functional groups that is structurally different from the isoelectric material, thereby forming an isoelectric hydrogel having a pI value in the range of 11.5<pI<14 on a hydrolytically stable substrate. In a related embodiment the isoelectric material which is at a concentration sufficiently high to set the pH of its aqueous solution equal to its pI value, is reacted with: (i) a cross-linker substantially free of ionic functional groups, and (ii) a hydrophilic polymer substantially free of ionic functional groups that is structurally different from the isoelectric material and the cross-linker.

In another aspect the invention provides a method of forming a hydrolytically stable isoelectric membrane having a pI value in the 11.5<pI<14 range, comprising the steps of (a) selecting a carbohydrate-based or polyhydroxy compound-based precursor having a hydroxyl group with a pKa value in the range of 11.5<pKa<14 and having an additional reactive group, (b) selecting a derivatizing agent having a substantially permanently cationic functional group and an additional reactive group, wherein the derivatizing agent is structurally different from the precursor, (c) selecting a concentration of the precursor and the derivatizing agent to establish a desired pI value, (d) selecting a cross-linker having two or more reactive groups, wherein the cross-linker being substantially free of ionic functional groups and structurally different from both the precursor and the derivatizing agent, (e) reacting the precursor, and the derivatizing agent with the cross-linker, thereby forming an isoelectric hydrogel having a pI value in the range of 11.5<pI<14 on a hydrolytically stable substrate. In another embodiment the method of forming a hydrolytically stable isoelectric membrane having a pI value in the 11.5<pI<14 range may further comprise the steps of (f) selecting a hydrolytically stable, hydrophilic polymer having at least two reactive groups that is substantially free of ionic functional groups and structurally different from both the precursor, the derivatizing agent and the cross-linker; and (g) reacting the hydrolytically stable, hydrophilic polymer, with the precursor, the derivatizing agent and the cross-linker.

Finally the invention provides for the use of hydrolytically stable isoelectric materials and membranes comprising hydrolytically stable isoelectric material for an isoelectric trapping separation of an ampholytic compound.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of the invention.

Other objects, advantages and embodiments of the invention will be apparent from the detailed description of the invention that follows.

### DETAILED DESCRIPTION OF THE INVENTION

Definitions

Unless defined otherwise, all technical and scientific terms used herein generally have the same meaning as commonly understood by one of ordinary skill in the art to which this invention belongs. Generally, the nomenclature used herein and the laboratory procedures described below are those well known and commonly employed in the art.

Reactive group, of a “functional group” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Exemplary functional groups include, but are not limited to hydroxyl, aldehyde, epoxy, halo, alkylsulfonyl, and arylsulfonyl groups and the like. Methods to prepare each of these functional groups are well known in the art and their application to or modification for a particular purpose is within the ability of one of skill in the art (see, for example, Sandier and Karo, eds. ORGANIC FUNCTIONAL GROUP PREPARATIONS, Academic Press, San Diego, 1989).

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂,—S(O)—CH₃, —CH₂—CH₂—S(O)₂—CH₃, —CH═CH—O—CH₃, —Si(CH₃)₃, —CH₂—CH═N—OCH₃,and —CH═CH—N (CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′—represents both —C(O)₂R′—and —R′C(O)₂—.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

The expression “having a pI value in the range of 11.5<pI<14” or “a pI value in the 11.5<pI<14 range” refers to an isoelectric point value in a range that at its lower extreme encompasses pI values of 11.5, 12, 12.5, 13, or 13.5, and at its higher extreme encompasses pI values ranging between 12, 12.5, 13, 13.5, or 14. Similarly, the expression “a pKa value in the range of 11.5<pI<14” and its equivalents, refers to pKa values in a range that at the lower extreme encompass pKa values of 11.5, 12, 12.5,13, or 13.5, and at the higher extreme encompass pKa values of 12, 12.5, 13, 13.5, or 14.

The term “isoelectric point” refers to the point at which a molecule or compound, which can exist in forms bearing either negative and/or positive charges, is electrically balanced, such that the net charge on the molecule or compound is zero. Thus, an isoelectric hydrogel material, or an isoelectric membrane is an entity that is electrically neutral or uncharged under specified conditions.

The term “ionic functional group” refers to refers to functional group that is capable of being ionized, such that the functional group may become either positively or negatively charged upon ionization.

The term “cationic functional group” refers to an ionic functional group, as defined above, that bears a positive charge under a given set of conditions. The phrase “permanently cationic functional group” refers to a molecule or compound that exists under defined conditions as a positively charged molecule at least more than about 90% of the time, preferably at least more than about 91%, 92%, 93%, 94% of the time, or more preferably at least more than about 95%, 96%, 97%, 98% or 99% of the time. An exemplary “permanently cationic functional group” is a quaternary ammonium group.

The phrase “substantially free of ionic groups” refers to a molecule that is at least more than about 90% free of ionic functional groups, preferably at least more than about 91%, 92%, 93%, 94% free of ionic functional groups, or more preferably at least more than about 95%, 96%, 97%, 98% or 99% free of ionic functional groups and most preferably is 100% free of ionic functional groups.

Introduction

In membrane-mediated isoelectric focusing and isoelectric trapping separations isoelectric hydrogels and/or membranes may serve either as ion-permeable separative barriers or ion-permeable cathodic electrode compartment barriers. Isoelectric hydrogels and/or membranes can play multiple roles to effect or aid the desired separations through buffering and/or sieving ability and/or through the prevention of convective mixing. Typically, the ion-permeable barriers, hydrogels and/or membranes are prepared from acrylamide and/or acrylamido weak and/or strong electrolyte derivatives by polymerization of suitable monomers, modifiers and crosslinkers.

Though acrylamide-based ion-permeable barriers, hydrogels and/or membranes have many outstanding and desirable characteristics, neither the monomers, nor the polymers are hydrolytically stable at above about pH 10.5. Hydrolysis of the amide bond creates weak acid functional groups in the polyacrylamide-based matrix which, upon dissociation, act as immobilized negative charges in the gel matrix, which create electroosmotic flow and/or bind certain analytes, both of which are often detrimental to the separation.

In addition to the limited hydrolytic stability of the acrylamide-based hydrogels and/or membranes in high pH environments, the formation of pI>10.5 isoelectric hydrogels and/or membranes is hindered by the lack of suitable acrylamido weak base derivatives with pK_{b} values between 2 and 4.

Since it is often desirable to utilize hydrogels and/or membranes that have pI values above 10.5, there is clearly a need in the art for hydrolytically stable, high-pI hydrogel and/or membrane compositions.

Fortunately, molecular configurations that result in isoelectric substances with a high pI value have been discovered, and are provided by the present invention. Such isoelectric materials can be used to create hydrolytically stable hydrogel materials that can further be used to create hydrolytically stable isoelectric membranes. Thus, the invention provides hydrolytically stable, high-pI hydrogel and/or membrane compositions for the separation of ampholytic compounds.

Chemistry of High pI Isoelectric Materials

The pKₐ values of the secondary alcohol groups of certain carbohydrates and polyhydroxy compounds lie in the 10.5<pKₐ range (e.g., 10.55, 10.6, 11, 11.5, 12, 12.5, 13, 13.5, 14), while the pKₐ values of the primary alcohol groups are typically in the pKₐ>13 range. For example, the pKa values a few common carbohydrates are as follows (Bruggink, C., AVH Association—7^{th} Symposium—Reims, March 2000 3-9; Masuda, T., et al., J. Chromatogr A., 961 (1) 89-96; Lee, Y-H. and Lin, T., Electrophoresis, 17, 333-340; Rong, D. and D'Souza, V., Tetrahedron Letters, 31 (30) 4275-4278, all incorporated herein by reference):

| Compound | pKₐ |
|---|---|
| Maltose | 11.94 |
| Lactose | 11.98 |
| Fructose | 12.03 |
| Mannose | 12.08 |
| Xylose | 12.15 |
| Glucose | 12.28 |
| Galactose | 12.39 |
| Dulcitol | 13.43 |
| Sorbitol | 13.60 |
| α-Methyl glucoside | 13.71 |
| 1,3-dimethoxy glycerol | 13.68 |
| γ-cyclodextrin | 12.05 (secondary OH) |
| β-cyclodextrin | 12.20 (secondary OH) |
| α-cyclodextrin | 12.33 (secondary OH) |

It has now been discovered that an isoelectric substance with a high pI value can be created from a molecules containing any of the following combinations: (i) at least one secondary OH group with a 11.5<pKₐ<14 (e.g., with a pKa of between about 11.5, 12, 12.5, 13, 13.5 and 14) and an amine group with a 1<pK_{b}<4 (e.g., with a pKb of between 1, 1.5, 2, 2.5, 3, 3.5, and 4); (ii) at least two secondary OH groups with pKₐ values in the 11.5<pKₐ<14 range and one strongly basic functional group (such as a quaternary ammonium group); or (iii) at least one secondary OH group with a pKₐ value in the 11.5<pKₐ<14 range and one such primary OH group with a pKₐ value in the 13<pKₐ range (e.g., with a pKa of about 13, 13.5, or 14) and one permanently cationic functional group (such as a quaternary ammonium group). After obtaining such an isoelectric material, the material can be used to produce a hydrogel and/or membrane by crosslinking it with any suitable bifunctional or polyfunctional agent or by grafting it onto any suitable crosslinkable or crosslinked substrate.

Secondary alcohols of many oligo- and polysaccharides including, but not restricted to, cyclodextrins, maltodextrins, amyloses, starches, dextrans, celluloses, luteoses, curdlans, guar gums, agaroses, etc., have the same desired property as the molecules described above (secondary OH groups with pKₐ values in the 10.5<pKₐ<14 range, primary OH groups in the 13<pKₐ range). These oligomers and/or polymers can be modified with suitable amine or quaternary ammonium functional groups to create high pI isoelectric materials which can be converted, e.g., by crosslinking, into suitable hydrogels and/or membranes.

Secondary alcohol groups of many oligomeric and polymeric materials including, but not restricted to, poly(vinylalcohol) and its derivatives, partially or fully hydrolyzed poly(epihalohydrine)s and their derivatives, partially or fully hydrolyzed poly(epihalohydrine-co-ethylene oxide)s and their derivatives polymers formed from polyhydroxy compounds and di-, oligo- or polyepoxides also have the same desired property (secondary OH groups with pKₐ values in the 10.5<pKₐ<14 range and primary OH groups with pKₐ values in the 13<pKₐ range) that makes them suitable for use in synthesizing an isoelectric substance with a high pI value. These oligomers and/or polymers can be modified with suitable amino or quaternary ammonium functional groups to create high-pI isoelectric materials which can be converted, e.g., by crosslinking, into suitable hydrogels and/or membranes.

The hydrophilic, polymeric nature of such hydrogels and/or membranes reduces the magnitude of electroosmotic flow through such hydrogels and/or membranes, which is very desirable for electrophoretic separations.

By varying the connection of the OH group-containing material and/or the type and/or the concentration of the crosslinking agent and the cationic functional group, the present invention can produce high-pI isoelectric hydrogels and/or membranes that can also act as sieving matrices in electrophoretic separations, similar to the way acrylamide-based gels do.

It will be appreciated that many additional tasks can be solved utilizing the hydrolytically stable, high-pI isoelectric hydrogel and/or compositions of the present invention without departing from the essence of this disclosure.

Apparatus Suitable for Use of High pI Isoelectric Materials

A membrane-based electrophoresis apparatus particularly suitable for isoelectric focussing or isoelectric trapping has been developed by The Texas A&M University System and Gradipore Limited (WO 02/24314, which is incorporated herein by reference). The apparatus, termed herein as “the Twinflow unit” comprises (a) a first electrolyte reservoir and a second electrolyte reservoir; (b) a first sample reservoir and a second sample reservoir; (c) a separation unit having a first electrolyte chamber in fluid connection with the first electrolyte reservoir, a second electrolyte chamber in fluid connection with the second electrolyte reservoir, a first sample chamber positioned between the first electrolyte chamber and the second electrolyte chamber, a second sample chamber positioned adjacent to the first sample chamber and between the first electrolyte chamber and the second electrolyte chamber, the first sample chamber being in fluid connection with the first sample reservoir, and the second sample chamber being in fluid connection with the second sample reservoir; (d) a first ion-permeable barrier positioned between the first sample chamber and the second sample chamber, the first ion-permeable barrier prevents substantial convective mixing of contents of the first and second sample chambers; (e) a second ion-permeable barrier positioned between the first electrolyte chamber and the first sample chamber, the second ion-permeable barrier prevents substantial convective mixing of contents of the first electrolyte chamber and the first sample chamber; (f) a third ion-permeable barrier positioned between the second sample chamber and the second electrolyte chamber, the third ion-permeable barrier prevents substantial convective mixing of contents of the second electrolyte chamber and the second sample chamber; (g) electrodes positioned in the first and second electrolyte chambers; (h) means for supplying electrolyte from the first electrolyte reservoir to the first electrolyte chamber, and from the second electrolyte reservoir to the second electrolyte chamber; and (i) means for supplying sample or liquid from at least the first sample reservoir to the first sample chamber, or from the second sample reservoir to the second sample chamber.

In use, a sample to be treated is placed in the first and/or second sample reservoirs and provided to, or circulated through, the first and/or second chambers. Electrolyte is placed in the first and second electrolyte reservoirs and passed to, or circulated through, the respective first and second electrolyte chambers without causing substantial mixing between the electrolyte in the two electrolyte reservoirs. Electrolyte or other liquid can be placed in first and/or second sample reservoirs if required. An electric potential is applied to the electrodes wherein one or more components in the first and/or second sample chamber are caused to move through a diffusion barrier to the second and/or first sample chamber, or to the first and/or second reservoir chambers. Treated sample or product can be collected in the second and/or first sample reservoir.

### EXAMPLES

Various hydrogels were prepared at temperatures ranging from room temperature to about 80° C. and reaction times varying from a few minutes to several days. Higher temperatures were used in order for the reaction to proceed at a reasonable rate. It has been found, however, that the actual reaction temperature and time of reaction incubation are not particularly critical to develop various hydrogels according to the present invention. It will be appreciated that as temperatures are elevated, the rate of reaction will increase and incubation times will be shorter. Accordingly, one of skill in the art will be able to choose appropriate reaction conditions and the determine how long the reaction needs to proceed. The following examples are meant to illustrate, but not limit the invention.

### Example 1

Weigh a 100 ml beaker. Place the weighed 100 ml beaker and two 230×190 ×6 mm, clean glass plates into a drying oven at 80° C. Cut a 160×200 mm piece of a Grade BFN 3 Papylon PVA paper (Sansho Co., Ltd, The 2nd Kitahama Building 1-29, Kitaham-Higashi, Chuoh-Ku, Osaka, Japan). Fit a 250 ml, two-neck, round bottom flask with a condenser and a nitrogen purge line. Place a 1″ football-shaped stir bar into the flask. Purge the system with nitrogen gas. Circulate ice-water through the condenser.

Place the flask into a heating mantle. Add 60 ml deionized water to the flask. Add 6.58 g (0.1645 mol) NaOH to the flask. Stir and heat the solution to a boil. Add 12 g (0.2727 mol secondary OH group equivalent) 99% hydrolyzed poly(vinylalcohol), average molecular weight 89,000-98,000 (PVA) to the flask. Maintain a nitrogen atmosphere over the reaction mixture, continue stirring and heating until PVA is completely dissolved. Turn off the heating mantle. Add 1.8 g (0.012 mol) glycidyl trimethylammonium chloride (Q) to the reaction mixture and stir until Q is dissolved.

Take the hot, bottom glass plate from the oven and place it onto a layer of paper towels. Take the hot, 100 ml beaker from the oven and weigh into it a 60 g aliquot of the hot, viscous reaction mixture. Quickly add to it 4.5 ml (4.916 g, 0.024 mol) glycerol diglycidyl ether and mix it well (manually) with a spatula. Pour half of the beaker's content onto the hot, bottom glass plate and quickly distribute the mixture over the plate by tilting it around. Lower the BFN 3 PVA substrate onto the reaction mixture and saturate the substrate with the reaction mixture.

Take the hot, cover glass plate from the oven, pour the second half of the reaction mixture from the beaker onto it and quickly distribute the mixture over the plate by tilting it. Lower the coated face of the cover plate onto the BFN 3 PVA substrate and press the plate to evenly distribute the reaction mixture over the entire surface of the BFN 3 PVA substrate. Place two 16×16×2″ cement patio paving stones onto the glass plates to compress them and squeeze out the excess reaction mixture.

Two hours later, remove the stones from the glass plates. Let the glass plate mold stand undisturbed at room temperature for 38 hours (total curing time 40 hours).

Fill a 16×12×6″ polypropylene tub with deionized water. Using a razor blade, cut along all four edges of the glass plate mold to remove the solidified, spilled-out reaction mixture. Lower the mold into the deionized water in the tub. Gently pull the glass plates apart under the water. The membrane should slip off easily from the glass plates. Gently slosh around the membrane in the water for about five minutes. Replace the water, slosh around the membrane for another five minutes. Repeat the procedure at least five times. Test the pH of the last wash water, it should be neutral. The salvage edge of the membrane should be clear, transparent, the surface of the membrane strong, even and slippery.

Store the membrane in deionized water in the fridge until used. The membrane will swell to a final thickness of about 0.4 to 0.7 mm. Using a pair of scissors, cut the membrane to size to fit the separation cartridge of the Twinflow unit. Punch inlet and outlet holes into the membrane and assemble the cartridge. Leak test the Twinflow unit, then commence the separation. After use, rinse the membrane and dispose it as solid waste.

The new compositions permit the preparation of hydrolytically and mechanically stable, high-pI hydrogels and/or membranes that were not available prior to this invention.

Numerous other hydrophilic, hydrolytically stable, high-pI compositions can be created along the synthetic lines described above, and these are expected to work just as well as the examples described below.

Hydrolytically stable high-pI hydrogels and/or membranes as outlined above has been experimentally demonstrated as follows.

### Example 1.1

High-pI, clear hydrogels were prepared by crosslinking trimethylammonio-β-cyclodextrin with epichlorohydrin in the presence of NaOH, at 80° C.

### Example 1.2

High-pI, clear hydrogels were prepared by crosslinking trimethylammonio-β-cyclodextrin with glycerol diglycidyl ether in the presence of NaOH, at 80° C.

### Example 1.3

High-pI, clear hydrogels were prepared by crosslinking trimethylammonio-β-cyclodextrin and poly(vinylalcohol) with glycerol diglycidyl ether in the presence of NaOH, at 80° C.

### Example 1.4

High-pI, clear hydrogels were prepared by crosslinking β-cyclodextrin and poly(vinylalcohol) with glycerol diglycidyl ether in the presence of glycidyl trimethylammonium chloride and NaOH, at 80° C.

### Example 1.5

High-pI isoelectric membranes were prepared by casting, in a glass mold, an 80° C. reaction mixture of glycidyl trimethylammonium chloride, β-cyclodextrin, poly(vinylalcohol), glycerol diglycidyl ether and NaOH over a Papylon Grade 3 poly(vinylalcohol) substrate and reacting the mixture at 60° C. for 40 hours.

### Example 1.6

High-pI isoelectric membranes prepared in Example 1.5 above were tested in the Twinflow unit described above. The membranes were used in a single separation compartment configuration, as the cathodic membrane. The anodic membrane was a pI=3 polyacrylamide isoelectric membrane (Gradipore Limited, Australia). The anolyte was 50 mM benzenesulfonic acid (BSH), the catholyte 50 mM benzyltrimethylammonium hydroxyde (BzOH)and 950 mM NaOH, the separation compartment contained tyramine (Tyr, approximate pI=10), histidine (His, pI=7.5) and meta-aminobenzoic acid (MABA, approximate pI=3.9) as analytes. Leak-free seal was achieved, and MABA, His and Tyr were trapped for the duration of the 180 min run. Neither BSH, nor BzOH invaded the separation compartment. When the run was repeated with a 50 mM benzyltrimethylammonium hydroxyde solution as the catholyte, Tyr was lost to the cathode compartment within 15 min indicating that the pI value of the high-pI membrane was greater than 12.7.

### Example 1.7

High-pI, clear hydrogels were prepared by reacting trimethylammonio-guar gum with glycidyl trimethylammonium chloride and crosslinking it with glycerol diglycidyl ether in the presence of NaOH, at 80° C.

### Example 1.8

High-pI, clear hydrogels were prepared by reacting glycidyl trimethylammonium chloride with poly(vinylalcohol), and crosslinking it with glycerol diglycidyl ether, in the presence of NaOH, at 80° C.

### Example 1.9

High-pI isoelectric membranes were prepared by casting, in a glass mold, an 80° C. reaction mixture of glycidyl trimethylammonium chloride, poly(vinylalcohol), glycerol diglycidyl ether and NaOH over a Papylon Grade 3 poly(vinylalcohol) substrate and reacting the mixture at 60° C. for 24 hours.

### Example 1.10

The high-pI isoelectric membranes prepared in Example 1.9 above were successfully tested in the Twinflow unit, in single separation compartment configuration, as the cathodic membrane. The separation compartment contained Tyr (pI=10), His (pI=7.5) and MABA (pI=3.9) as analytes. Leak-free seal was achieved and MABA, His and Tyr were trapped for the duration of the 180 min run. When the run was repeated with a pH 12 NaOH solution as the catholyte, Tyr was lost to the cathode compartment, within 15 min, indicating that the pI value of the high pI membrane was greater than 12.

### Example 1.11

High-pI isoelectric membranes were prepared by casting, in a glass mold, an 80° C. reaction mixture of glycidyl trimethylammonium chloride, poly(vinylalcohol), glycerol diglycidyl ether and NaOH over a Papylon Grade 3 poly(vinylalcohol) substrate and reacting the mixture at room temperature for 40 hours.

### Example 1.12

The high-pI isoelectric membranes prepared in Example 1.11 above were successfully tested in the Twinflow unit, in single separation compartment configuration, as the cathodic membrane. The separation compartment contained Tyr (pI=10), His (pI=7.5) and MABA (pI=3.9) as analytes. Leak-free seal was achieved and MABA, His and Tyr were trapped for the duration of the 180 min run.

### Example 1.13

High-pI isoelectric membranes were prepared by casting, in a glass mold, an 80° C. reaction mixture of glycidyl trimethylammonium chloride, poly(vinylalcohol), glycerol diglycidyl ether and NaOH over a Papylon Grade 4 poly(vinylalcohol) substrate and reacting the mixture at room temperature for 40 hours.

High-pI isoelectric membranes prepared in Example 1.13 above were successfully tested in the Twinflow unit, in single separation compartment configuration, as the cathodic membrane. The separation compartment contained Tyr (pI=10) and MABA (pI=3.9) as analytes. Leak-free seal was achieved and both MABA and Tyr were trapped for the duration of the 180 min run.

### Example 1.15

High-pI isoelectric membranes were prepared by casting, in a glass mold, an 80° C. reaction mixture of glycidyl trimethylammonium chloride, poly(vinylalcohol), glycerol diglycidyl ether and NaOH over a Papylon Grade 2 poly(vinylalcohol) substrate and reacting the mixture at room temperature for 40 hours.

### Example 1.16

High-pI isoelectric membranes prepared in Example 1.15 above were successfully tested in the Twinflow unit, in single separation compartment configuration, as the cathodic membrane. The separation compartment contained Tyr (pI=10), His (pI=7.5) and MABA (pI=3.9) as analytes. Leak-free seal was achieved and both MABA and Tyr were trapped for the duration of the 180 min run.

### Example 1.17

High-pI isoelectric membranes prepared in Example 1.15 and Example 1.16 above were tested in the Twinflow unit, in single separation compartment configuration, as the cathodic membranes. The separation compartment contained a recombinant thyroid-stimulating hormone (rTSH) preparation in a growth medium with a conductivity of 5000 μS. Desalting of the sample was successfully completed to a residual conductivity of about 800 μS, and neither albumin, nor rTSH was lost indicating that the high-pI isoelectric membranes functioned properly as cathodic isoelectric membranes.

### Example 1.18

High-pI isoelectric membranes prepared according to Example 1.11 and Example 1.15 above were successfully tested as cathodic membranes in over 10 isoelectric trapping (IET) separations using the Twinflow unit. Each time, when the NaOH concentration in the catholyte was 200 mM or higher, and the IET current was sufficiently high, the membranes behaved satisfactorily.

It will be appreciated by persons skilled in the art that numerous variations and/or modifications may be made to the invention as shown in the specific embodiments without departing from the spirit or scope of the invention as broadly disclosed. The present embodiments are, therefore, to be considered in all respects as illustrative and not restrictive.

What is claimed is:

1. A hydrolytically stable isoelectric hydrogel material having a pI value in the range of 11.5<pI<14, wherein the hydrolytically stable isoelectric hydrogel material comprises:
(a) an isoelectric compound having a pI value in the range of 11.5<pI<14, wherein the pI is established by a substantially permanently cationic group and two hydroxyl groups with pKₐ values in the range of 11.5<pKₐ<14,
(b) the isoelectric compound having an additional reactive group, and
(c) a cross-linker substantially free of ionic functional groups and comprising two groups able to react with the isoelectric compound without altering the pI value of the isoelectric compound by more than about 1 pH unit,
wherein the pI value of the isoelectric hydrogel is conferred by the pI value of the isoelectric compound.

2. The hydrolytically stable isoelectric hydrogel material of claim 1, further comprising:
(d) a hydrolytically stable hydrophilic polymer, the polymer comprising two or more reactive groups and being substantially free of ionic functional groups, wherein the cross-linker is substantially free of ionic functional groups and comprising two groups able to react with both the isoelectric compound and the hydrolytically stable polymer without altering the pI value of the isoelectric compound by more than about 1 pH unit.

3. The isoelectric hydrogel material of claim 1, wherein the pI value of the isoelectric hydrogel is changed by changing identity of the isoelectric compound.

4. The isoelectric hydrogel material of claim 1, wherein the isoelectric compound is present at a concentration equal to or higher than necessary to create an aqueous solution of the isoelectric compound with a pH equal to the pI value of the isoelectric compound.

5. The hydrolytically stable isoelectric hydrogel material of claim 2, wherein:
the isoelectric compound is a native or derivatized carbohydrate or polyhydroxy compound;
the permanently cationic functional group is a quaternary ammonium group;
the additional reactive group of the isoelectric compound is a hydroxyl group,
the hydrolytically stable hydrophilic polymer is an oligosaccharide or a synthetic polymer containing multiple hydroxyl groups, and
the cross-linker has two or more, reactive groups selected from the group consisting of an aldehyde, epoxy, halo, alkylsulfonyl, and arylsulfonyl groups.

6. The hydrolytically stable isoelectric hydrogel material of claim 5, wherein:
the native or derivatized carbohydrate or polyhydroxy compound is selected from the group consisting of native or derivatized monosaccharides, native or derivatized disaccharides, native or derivatized trisaccharides, native or derivatized oligosaccharides, native or derivatized polysaccharides, native or derivatized cyclodextrins, native or derivatized maltodextrins, native or derivatized amyloses, native or derivatized dextrans, native or derivatized starches, native or derivatized celluloses, and native or derivatized guar gums,
the quaternary ammonium group comprises one or more substituents selected from the group consisting of methyl, ethyl, propyl, butyl, higher alkyl, hydroxymethyl, hydroxyethyl, hydroxypropyl, hydroxybutyl, higher hydroxyalkyl, saturated ring systems, and unsaturated ring systems,
the hydrolytically stable hydrophilic polymer is selected from the group consisting of hydroxymethyl cellulose, hydroxyethyl cellulose, and poly(vinyl alcohol), and
the cross-linker is selected from the group consisting of glutaraldehyde, glycerol-1,3-diglycidyl ether, bis-(2-bromoethylene)-ethyleneglycol, and bis(2-tosylethylene)-ethyleneglycol.

7. The hydrolytically stable isoelectric hydrogel material of claim 6, wherein:
the isoelectric material is a glycidyl trimethylammonium derivative of beta-cyclodextrin,
the hydrolytically stable hydrophilic polymer is poly(vinyl alcohol), and
the cross-linking agent is glycerol-1,3-diglycidyl ether.

8. A hydrolytically stable isoelectric membrane having a pI value in the range of 11.5<pI<14 comprising:
the hydrolytically stable isoelectric hydrogel of claim 1, supported on a hydrolytically stable, porous, inert or reactive substrate.

9. The hydrolytically stable isoelectric hydrogel membrane of claim 8, wherein the porous substrate is selected from the group consisting of poly(vinyl alcohol), partially or fully hydrolyzed poly(epihalohydrin), partially or fully hydrolyzed poly(epihalohydrin-co-ethylene oxide), poly(vinyl sulfone), and poly(ether-ether ketone).

10. A hydrolytically stable hydrophilic isoelectric hydrogel material having a pI value in the range of 11.5<pI<14 comprised of:
(a) a precursor comprising a hydroxyl group with a pKa value in the range of 11.5<pKₐ<14 and an additional reactive group,
(b) a derivatizing agent comprising a permanently cationic functional group and an additional reactive group, and
(c) a cross-linker substantially free of ionic functional groups and comprising two groups able to react with the isoelectric compound without altering the pI value of the isoelectric compound by more than about 1 pH unit,
wherein the derivatizing agent is structurally different from the precursor,
wherein the pI value of the isoelectric hydrogel is conferred by the concentration of the derivatizing agent and the precursor and the pKa value of the hydroxyl group of the precursor, and
wherein the cross-linker is structurally different from both the precursor and the derivatizing agent.

11. The hydrolytically stable hydrophilic isoelectric hydrogel material of claim 10, further comprising:
(d) a hydrolytically stable hydrophilic polymer having two or more reactive groups that is substantially free of ionic functional groups, wherein the hydrophilic polymer is structurally different from the precursor and the derivatizing agent;
wherein the cross-linker is substantially free of ionic functional groups and having two groups able to react with both the precursor and the hydrolytically stable hydrophillic polymer.

12. The hydrolytically stable hydrophilic isoelectric hydrogel material of claim 11, wherein:
the precursor is a native or derivatized carbohydrate or polyhydroxy compound,
the derivatizing agent comprises a quaternary ammonium group,
the reactive group of the derivatizing agent is selected from the group consisting of hydroxyl, aldehyde, epoxy, halo, alkylsulfonyl, and arylsulfonyl groups,
the hydrolytically stable hydrophilic polymer is an oligosaccharide or a synthetic polymer containing multiple hydroxyl groups, and
the cross-linker has two or more reactive groups selected from the group consisting of aldehyde, epoxy, halo, alkylsulfonyl, and arylsulfonyl groups.

13. The hydrolytically stable hydrophilic isoelectric hydrogel material of claim 12, wherein:
the precursor is selected from the group consisting of native or derivatized monosaccharides, native or derivatized disaccharides, native or derivatized trisaccharides, native or derivatized oligosaccharides, native or derivatized polysaccharides, native or derivatized cyclodextrins, native or derivatized maltodextrins, native or derivatized amyloses, native or derivatized dextrins, native or derivatized starches, native or derivatized celluloses, and native or derivatized guar gums,
the quaternary ammonium group of the derivatizing agent contains one or more substituents selected from the group consisting of methyl, ethyl, propyl, butyl, higher alkyl, hydroxymethyl, hydroxyethyl, hydroxypropyl, hydroxybutyl, higher hydroxyalkyl groups, saturated ring systems, and unsaturated ring systems,
the cross-linker is selected from the group consisting of glutaraldehyde, glycerol-1,3-diglycidyl ether, bis-(2-bromoethylene)-ethyleneglycol, and bis(2-tosylethylene)-ethyleneglycol, and
the hydrolytically stable polymer is selected from the group consisting of hydroxyethyl cellulose, hydroxymethyl cellulose, and poly(vinyl alcohol).

14. The hydrolytically stable hydrophilic isoelectric hydrogel material of claim 13, wherein:
the precursor is beta-cyclodextrin,
the derivatizing agent is glycidyl-trimethylammonium chloride,
the cross-linker is glycerol-1,3-diglycidyl ether, and
the hydrolytically stable hydrophilic polymer is poly(vinyl alcohol).

15. The hydrolytically stable hydrophilic isoelectric hydrogel material of claim 13, wherein:
the precursor is poly(vinylalcohol), and
the cross-linker is glycerol-1,3-diglycidyl ether and the derivatizing agent is glycidyl-trimethylammonium chloride.

16. A hydrolytically stable isoelectric membrane having a pI in the range of 11.5<pI<14 comprising a hydrolytically stable isoelectric hydrogel having a pI in the 11.5<pI<14 range of claim 10, supported on a hydrolytically stable, porous, inert or reactive substrate.

17. The hydrolytically stable isoelectric membrane of claim 16, wherein the material of the porous substrate is selected from the group consisting of poly(vinyl alcohol), partially or fully hydrolyzed poly(epihalohydrin), partially or fully hydrolyzed poly(epihalohydrin-co-ethylene oxide), poly(vinyl sulfone), and poly(ether-ether ketone).

18. A method or roaming a hydrolytically stable isoelectric membrane having a pI value in the range of 11.5<pI<14, comprising the steps of:
(a) selecting a carbohydrate-based or polyhydroxy compound-based isoelectric material having a pI value in the 11.5<pI<14 range,
(b) reacting the isoelectric material at a concentration sufficiently high to set the pH of its aqueous solution equal to its pI value, with:
(i) a cross-linker substantially free of ionic functional groups that is structurally different from the isoelectric material,
thereby forming an isoelectric hydrogel having a pI value in the range of 11.5<pI<14 on a hydrolytically stable substrate.

19. The method of forming a hydrolytically stable isoelectric membrane of claim 18, further comprising the step of:
(c) reacting the isoelectric material at a concentration sufficiently high to set the pH of its aqueous solution equal to its pI value, with:
(i) a cross-linker substantially free of ionic functional groups, and
(ii) a hydrophilic polymer substantially free of ionic functional groups that is structurally different from the isoelectric material and the cross-linker.

20. A method of forming a hydrolytically stable isoelectric membrane having a pI value in the 11.5<pI<14 range, comprising the steps of:
(a) selecting a carbohydrate-based or polyhydroxy compound-based precursor having a hydroxyl group with a pKa value in the range of 11.5<pKa<14 and having an additional reactive group,
(b) selecting a derivatizing agent having a substantially permanently cationic functional group and an additional reactive group, wherein the derivatizing agent is structurally different from the precursor,
(c) selecting a concentration of the precursor and the derivatizing agent to establish a desired pI value,
(d) selecting a cross-linker having two or more reactive groups, wherein the cross-linker being substantially free of ionic functional groups and structurally different from both the precursor and the derivatizing agent,
(e) reacting the precursor, and the derivatizing agent with the cross-linker,
thereby forming an isoelectric hydrogel having a pI value in the range of 11.5<pI<14 on a hydrolytically stable substrate.

21. The method of forming a hydrolytically stable isoelectric membrane having a pI value in the 11.5<pI<14 range of claim 20, further comprising the steps of:
(f) selecting a hydrolytically stable, hydrophilic polymer having at least two reactive groups that is substantially free of ionic functional groups and structurally different from both the precursor, the derivatizing agent and the cross-linker; and
(g) reacting the hydrolytically stable, hydrophilic polymer, with the precursor, the derivatizing agent and the cross-linker,
thereby forming an isoelectric hydrogel having a pI value in the range of 11.5<pI<14 on a hydrolytically stable substrate.

22. A hydrolytically stable isoelectric membrane having a pI value in the range of 11.5<pI<14 produced by the method of claim 18.

23. A hydrolytically stable isoelectric membrane having a pI value in the 11.5<pI<14 range produced by the method of claim 20.

24. Use of a hydrolytically stable isoelectric membrane having a pI value in the range of 11.5<pI<14 according to any one of claims 16, 17, 22 or 23 for an isoelectric trapping separation of an ampholytic compound.
